SEQUENCE LISTING (1 sequence)

SEQ ID NO: 1 - DNA, 1584 bp, Artificial Sequence
  SPARC coding sequence
gtaatcctagcactttgggaagccaaggcattcggattgcccaagctcaggagttcgagaccagcctgggcaacatgttgaaaccccatttctactaaaaatacaataaattagctgggtgttgtggcatgtgcgcctgtaatcccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccggaaaccacaaaactttttgaggacaaggaccaggtatttattaattctcatacctcccagagtgttaggcacaaaataaacattcaaccaagacctgttgcactgagcagttcatatataacaggagtgacccaagttgaaacgtagaatcagccctctcataccactttttgccaggtgatcataggcaagttacttagcatctatgtttccttattattaaaatggtcataattacaatgcctaagataaggggttgctgtgaagattattaaatcctcagtaaactttggctattgttactcctatgattatcatcaatatcatcaattccttatctgttcaatactggtggcacaggtccaccagctagatgtctaatcccttatgtgtctattagtggtacaagtggagtttgagtgggatttttttttttaagaccagttccaaatcatcaaggatgataccacagtagcagcttgtcttgtctgtacagtggtaagtcctggccttgcctttgtggcaaatacaacccccttgaattgcttggcccttctcagcattgcctaatattagggaggactcctgtaaagctcactggtagaagatcaagacacttgggcctggttctgcccctgggggccattgggtaattccttgcagtctccaggcctcacttgccctctgaacaagaaagaggctgttctgggtcatccctccaggcctgtccagcctggcactctgtgagtcggtttaggcagcagccccggaacagatgaggcaggcagggttgggacgtttggtcaggacagcccaccgcaaaaagaggaggaaagaaatgaaagacagagacagctttggctatgggagaaggaggaggccgggggaaggaggagacaggaggaggagggaccacggggtggaggggagatagacccagcccagagctctgagtggtttcctgttgcctgtctctaaacccctccacattcccgcggtccttcagactgcccggagagcgcgctctgcctgccgcctgcctgcctgccactgaggtatgtgtgacccccgcccagcctttcccttctatagttgcacgttgcaccaaccccgacacccccgttcacgccgtcagctcgtgtgcaagggagggaagctctgctgaggatgcgcctctcctcccggctccatcacggctccccttaagagcatggccctcggtcctgtctgcctgttgcttttcagaaggtggactcactgtgtaactttgtcttcccttacaggtt

(19) United States
(12) Patent Application Publication
(10) Pub. No.: US 2010/0286058 A1
(43) Pub. Date: Nov. 11, 2010

(54) Assessing Chemotherapy Resistance of Colorectal Tumors by Determining Sparc Hypermethylation

(76) Inventor: Isabella Tai, Vancouver (CA)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/530,594

(22) PCT Filed: Mar. 7, 2008
(86) PCT No.: PCT/CA2008/000458

§ 371 Date: Jul. 10, 2010

Related U.S. Application Data
(60) Provisional application No. 60/905,806, filed on Mar. 9, 2007.

(51) Int. Cl. A61K 38/02; C12Q 1/68; C40B 30/00; A61K 31/706; A61K 31/245; A61K 31/7068; A61P 35/00

(52) U.S. Cl. 514/19.3; 435/6; 506/7; 514/43; 514/535; 514/49

(57) ABSTRACT

Hypermethylation of the SPARC promoter is identified as a mechanism for repressing SPARC gene in cancer resulting in resistance to therapy. The restoration of SPARC expression with demethylating agents, such as 5-Aza-2′deoxycytidine, is shown to enhance chemosensitivity. The invention provides a mechanism of limiting the number of patients exposed to toxicity of demethylating agents by targeting its administration to the subset of patients with hypermethylation of the SPARC promoter.

### CROSS-REFERENCE TO RELATED APPLICATIONS

This patent application claims the benefit of U.S. Provisional Patent Application No. 60/905,806, filed Mar. 9, 2007, which is hereby incorporated by reference in its entirety.

### BACKGROUND OF THE INVENTION

Resistance to chemotherapy is common to many types of cancer and contributes to the high mortality rates in cancer patients. Many factors can play a part in the initial intrinsic resistance to therapy, such as the upregulation of efflux pumps from multidrug resistance (MDR) family P-glycoprotein and other MDR proteins (for example multi-drug resistance-associated protein MRP). Such efflux pumps remove chemotherapeutic agents and their metabolites out of cells, thereby decreasing the efficacy of the chemotherapeutic regimen. However, not all cancers that are resistant to chemotherapy have high expression levels of MDR proteins, suggesting that there may be other mechanisms for therapeutic resistance.

Some cancers are particularly resistant to therapies due to genetic changes. For example, colorectal cancer (CRC), often accumulates numerous genetic mutations in the progression to tumorigenesis that can also contribute to drug resistance (mutations in p53; loss of DNA mismatch repair (MMR) genes associated with an increased rate of mutation to drug resistance in hereditary non-polyposis colorectal cancer (HNPCC); K-ras mutations; loss of heterozygosity of chromosomes 18q and 22q; and mutations in cell cycle regulatory genes, p21 and p27, is associated with preventing apoptosis in tumors following some chemotherapeutic treatments).

Another recently recognized mechanism of chemo-resistance is the under-expression of the secreted protein, acidic, rich in cysteine (SPARC) protein (Tai et al. J. Clin. Invest. (2005) 115(6): 1492-1502). SPARC, also known as osteonectin, BM-40 and 43K protein, is a type of extracellular protein, termed a matricellular protein, having counter-adhesive properties shown to disrupt cell-matrix interactions. SPARC is a Ca2+-binding glycoprotein, which maps to chromosome 5q31-q33, the genomic sequence is reported to have 10 exons and 9 introns with the cDNA extending over ˜3 kb. Representative Homo sapiens SPARC sequences are listed in GenBank under accession numbers NM_{—}003118.2 (gi48675809-mRNA), J03040 and J02863. A representative sequence containing the SPARC promoter, submitted by Jendraschak and Kaminski (Genomics (1998) 50(1):53-60), is U65081.1 (gi3253140) containing 1370 bp. The SPARC promoter region (accession number X82259) was characterized using various promoter constructs in a luciferase assay by Hafner M. et al. (Matrix Biology (1994) 14:733-741).

SPARC has been associated with bone mineralization tissue remodeling, endothelial cell migration, apoptosis, angiogenesis, scleroderma and adipose tissue accumulation. SPARC has also been associated with poor overall survival in head and neck cancer (HNSCC) patients (Chin D. et al. Int. J. Cancer (2005) 113:789-797), poor prognosis in human breast cancer (Jones C. et al. Cancer research (2004) 64:3037-3045), tumor suppressor activity in human ovarian carcinoma cells (Mok S C. et al. Oncogene (1996) 12:1895-1901), development of squamous cell carcinomas in mice (Aycock R L. et al. J. Invest. Dermatol. (2004) 123:592-599), inhibition of Lewis lung carcinoma (LLC) cells (Brekken R A. et al. J. Clin. Invest. (2003) 111:487-495) and advanced stage human gastric carcinomas (Wang C-S. et al. British Journal of Cancer (2004) 91:1924-1930).

Promoter hypermethylation is recognized to play an important role in repressing expression of the gene under the control of the hypermethylated promoter. Chemotherapeutic agents are known in the art which can reverse promoter hypermethylation, but these agents often have significant toxicity. The invention provides a mechanism of limiting the number of patients exposed to toxicity of demethylating agents by targeting its administration to the subset of patients with various forms of SPARC promoter hypermethylation.

### BRIEF SUMMARY OF THE INVENTION

The invention provides methods for assessing the resistance to chemotherapy of a tumor in a mammal comprising:
(a) obtaining tissue from the tumor;
(b) determining the level of SPARC promoter methylation in the tumor tissue; and
(c) assessing the resistance to chemotherapy of the tumor,
   wherein the resistance to chemotherapy of the tumor is directly proportional to the level of SPARC promoter methylation in the tumor tissue.

In another embodiment, the invention provides methods for assessing the resistance to chemotherapy of a tumor in a mammal comprising:
(a) obtaining tissue from the tumor;
(b) determining the level of SPARC promoter methylation in the tumor tissue;
(c) obtaining corresponding normal tissue from a mammal,
(d) determining the level of SPARC promoter methylation in the corresponding normal tissue;
(c) assessing the resistance to chemotherapy of the tumor,
   wherein the resistance to chemotherapy of the tumor is directly proportional to the difference between the level methylation of the SPARC promoter in the tumor tissue and the level of methylation of the SPARC promoter in the corresponding normal tissue.

Further, the invention provides methods for assessing resistance to chemotherapy of the tumor wherein the level of resistance is directly proportional to the difference between the level methylation of the SPARC promoter in the tumor tissue and the level of methylation of the SPARC promoter in the corresponding normal tissue, and wherein SPARC promoter methylation determined by assessing methylation at the following cytosines in the SPARC promoter:

| | Nucleotide No. in |
|---|---|
| CpG No. in FIG. 1 | SEQ ID NO. 1 |
| 1 | 186 |
| 2 | 210 |
| 3 | 240 |
| 4 | 1099 |
| 5 | 1120 |
| 6 | 1184 |
| 7 | 1218 |
| 8 | 1296 |
| 9 | 1298 |
| 10 | 1315 |
| 11 | 1322 |
| 12 | 1324 |
| 13 | 1336 |
| 14 | 1372 |
| 15 | 1414 |
| 16 | 1423 |
| 17 | 1429 |
| 18 | 1432 |
| 19 | 1440 |
| 20 | 1485 |
| 21 | 1496 |
| 22 | 1521 |

In another embodiment the invention predicts resistance to chemotherapy in a mammal if at least 3 of the cytosines at the following positions are methylated in tumor tissue, but not the corresponding normal tissue:

| | Nucleotide No. in |
|---|---|
| CpG No. in FIG. 1 | SEQ ID NO. 1 |
| 1 | 186 |
| 2 | 210 |
| 8 | 1296 |
| 9 | 1298 |
| 10 | 1315 |
| 16 | 1423 |
| 17 | 1429 |
| 18 | 1432 |

In a further embodiment the invention predicts resistance to chemotherapy in a mammal if the cytosines at the following positions are methylated in tumor tissue:

| | Nucleotide No. in |
|---|---|
| CpG No. in FIG. 1 | SEQ ID NO. 1 |
| 1 | 186 |
| 8 | 1296 |
| 9 | 1298 |
| 16 | 1423 |

In a yet another embodiment the invention predicts resistance to chemotherapy in a mammal least 5 of the cytosines at the following positions are methylated in tumor tissue:

| | Nucleotide No. in |
|---|---|
| CpG No. in FIG. 1 | SEQ ID NO. 1 |
| 1 | 186 |
| 2 | 210 |
| 8 | 1296 |
| 9 | 1298 |
| 10 | 1315 |
| 16 | 1423 |
| 17 | 1429 |
| 18 | 1432 |

Suitable corresponding normal tissue can come from the same mammal as the mammal with the tumor or from a different mammal from the mammal with the tumor.

The level of methylation of the SPARC promoter can be determined using any suitable method including, for example, methylation sensitive PCR, methylation-sensitive restriction digestion, bisulfite modification amplified by fluorescence-based real-time quantitative PCR, hybridization to oligonucleotide microarrays, and binding to anti-methylcytosine antibodies.

The invention provides methods of treating a mammal with a tumor comprising:
(a) obtaining tissue from the tumor;
(b) determining the level of SPARC promoter methylation in the tumor tissue;
(c) obtaining corresponding normal tissue from a mammal,
(d) determining the level of SPARC promoter methylation in the corresponding normal tissue;
(c) assessing the resistance to chemotherapy of the tumor, wherein resistance to chemotherapy of the tumor is directly proportional to the level of SPARC promoter methylation in the tumor tissue, wherein determining the level of SPARC promoter methylation comprises determining methylation status of the cytosines at the following positions:

| | Nucleotide No. in |
|---|---|
| CpG No. in FIG. 1 | SEQ ID NO. 1 |
| 1 | 186 |
| 2 | 210 |
| 3 | 240 |
| 4 | 1099 |
| 5 | 1120 |
| 6 | 1184 |
| 7 | 1218 |
| 8 | 1296 |
| 9 | 1298 |
| 10 | 1315 |
| 11 | 1322 |
| 12 | 1324 |
| 13 | 1336 |
| 14 | 1372 |
| 15 | 1414 |
| 16 | 1423 |
| 17 | 1429 |
| 18 | 1432 |
| 19 | 1440 |
| 20 | 1485 |
| 21 | 1496 |
| 22 | 1521 |

and (e) administering a SPARC polypeptide and/or a demethylating agent to the mammal based on the level of SPARC promoter methylation in the tissue.

Alterative criteria provided by the invention for administering a SPARC polypeptide and/or a demethylating agent to a mammal with a tumor include:
(a) wherein at least 3 of the cytosines at the following positions are methylated in tumor, but not in the corresponding normal tissue:

| | Nucleotide No. in |
|---|---|
| CpG No. in FIG. 1 | SEQ ID NO. 1 |
| 1 | 186 |
| 2 | 210 |
| 8 | 1296 |
| 9 | 1298 |
| 10 | 1315 |
| 16 | 1423 |
| 17 | 1429 |
| 18 | 1432 |

(b) wherein at least 3 of the cytosines at the following positions are methylated in tumor tissue:

| | Nucleotide No. in |
|---|---|
| CpG No. in FIG. 1 | SEQ ID NO. 1 |
| 1 | 186 |
| 8 | 1296 |
| 9 | 1298 |
| 16 | 1423 |

or (c) wherein at least 5 of the cytosines in the following positions are methylated in tumor tissue:

| | Nucleotide No. in |
|---|---|
| CpG No. in FIG. 1 | SEQ ID NO. 1 |
| 1 | 186 |
| 2 | 210 |
| 8 | 1296 |
| 9 | 1298 |
| 10 | 1315 |
| 16 | 1423 |
| 17 | 1429 |
| 18 | 1432 |

### BRIEF DESCRIPTION OF THE SEVERAL VIEWS OF THE DRAWING(S)

FIG. 1 is a map of the CpG island in the promoter region 5′ of the SPARC coding sequence.

FIG. 2a shows the results of methylation Specific PCR of untreated cell lines.

FIG. 2b shows the results of methylation Specific PCR of cell lineswith 5-Aza for 7 days.

FIG. 2c shows ChIP assay results with amplified PCR products of the regions within the SPARC promoter and INK4A(p16) control promoter.

FIG. 2d shows an assessment of SPARC mRNA by RT-PCR of MIP101 cells after exposure to increasing concentrations of 5-Aza.

FIG. 3 shows methylation Specific PCR of the SPARC promoter from primary colorectal cancers and normal colon.

FIG. 4a shows a tabular summary of the frequency of methylation, partial methylation and the absence of methylation at each CpG site in tumor tissue versus normal colon. The results are based on the direct bisulfite DNA sequencing of human colorectal cancers and normal colon in the 1,500 bp region 5′ of the SPARC gene. (M=methylation, P=partial methylation, and U=unmethylation at each CpG site.)

FIG. 4b shows an example of a DNA sequencing chromatogram of an unmethylated CpG.

FIG. 4c shows an example of a DNA sequencing chromatogram of a partially methylated CpG.

FIG. 4d shows an example of a DNA sequencing chromatogram of a methylated CpG.

FIG. 4e shows the distribution of unmethylated, partially methylated, and methylated CpG sites in normal colon versus tumor (CRC).

FIG. 5a shows the effect of exposing CRC cells to 5-Aza, a demethylating agent, on their sensitivity to 5-FU.

FIG. 5b shows the effect of exposing CRC cells to 5-Aza, a demethylating agent, on their sensitivity to 5-FU in a colony assay.

FIG. 5c shows results with a Caspase 3/7 apoptosis assay.

FIG. 5d shows results with a TUNEL apoptosis assay.

FIG. 5e shows results of a TUNEL apoptosis assay of MIP101 cells demonstrating enhanced sensitivity to 5-FU in cells pre-incubated 5-Aza.

FIG. 6a shows the effect on proliferation of the exposure of CRC cell lines to 5-Aza.

FIG. 6b is a tabular summary showing the effect on proliferation of the exposure of CRC cell lines to 5-Aza.

FIG. 6c shows the effect on proliferation of the exposure of CRC cell lines to 5-Aza and 5-FU.

FIG. 6d is a tabular summary showing the effect on proliferation of the exposure of CRC cell lines to to 5-Aza and 5-FU.

FIG. 7a shows the effect of 5-Aza on SPARC RNA expression in MIP cells.

FIG. 7b shows the effect of 5-Aza on SPARC protein expression in MIP cells.

### DETAILED DESCRIPTION OF THE INVENTION

As used herein, the term “cancer” refers to a proliferative disorder caused or characterized by the proliferation of cells which have lost susceptibility to normal growth control. The term cancer, as used in the present application, includes tumors and any other proliferative disorders. Cancers of the same tissue type usually originate in the same tissue, and may be divided into different subtypes based on their biological characteristics. Four general categories of cancers are carcinoma (epithelial tissue derived), sarcoma (connective tissue or mesodermal derived), leukemia (blood-forming tissue derived) and lymphoma (lymph tissue derived). Over 200 different types of cancers are known, and every organ and tissue of the body may be affected. Specific examples of cancers that do not limit the definition of cancer may include melanoma, leukemia, astrocytoma, glioblastoma, retinoblastoma, lymphoma, glioma, Hodgkins' lymphoma and chronic lymphocyte leukemia. Examples of organs and tissues that may be affected by various cancers include pancreas, breast, thyroid, ovary, uterus, testis, prostate, thyroid, pituitary gland, adrenal gland, kidney, stomach, esophagus or rectum, head and neck, bone, nervous system, skin, blood, nasopharyngeal tissue, lung, urinary tract, cervix, vagina, exocrine glands and endocrine glands. Alternatively, a cancer may be multicentric or of unknown primary site (CUPS).

As used herein, a ‘cancerous cell’ refers to a cell that has undergone a transformation event and whose growth is no longer regulated to the same extent as before said transformation event. A tumor refers to a collection of cancerous cells, often found as a solid or semi-solid lump in or on the tissue or a patient or test subject.

A cancer or cancerous cell may be described as “sensitive to” or “resistant to” a given therapeutic regimen or chemotherapeutic agent based on the ability of the regimen to kill cancer cells or decrease tumor size, reduce overall cancer growth (i.e. through reduction of angiogenesis), and/or inhibit metastasis. Cancer cells that are resistant to a therapeutic regimen may not respond to the regimen and may continue to proliferate. Cancer cells that are sensitive to a therapeutic regimen may respond to the regimen resulting in cell death, a reduction in tumor size, reduced overall growth (tumor burden) or inhibition of metastasis. For example, this desirably manifest itself in a reduction in tumor size, overall growth/tumor burden or the incidence of metastasis of about 10% or more, for example, about 30%, about 40%, about 50%, about 60%, about 70%, about 80%, or more, to about 2-fold, about 3-fold, about 4-fold, about 5-fold, about 10-fold, about 15-fold, about 20-fold or more. Monitoring of a response may be accomplished by numerous pathological, clinical and imaging methods as described herein and known to persons of skill in the art.

As used herein, a “therapeutic regimen” or “therapy” refers to the administration of at least one agent which is harmful to cancerous cells. Suitable therapeutic regimens for use in accordance with the invention include, but are not limited to, “chemotherapeutic regimens,” “radiotherapeutic regimens,” “alternative therapeutic regimen” and combinations thereof.

As used herein, a “chemotherapeutic regimen” or “chemotherapy” refers to the administration of at least one chemotherapy agent which is harmful to destroy cancerous cells. There are a myriad of such chemotherapy agents available to a clinician. Chemotherapy agents may be administered to a subject in a single bolus dose, or may be administered in smaller doses over time. A single chemotherapeutic agent may be used (single-agent therapy) or more than one agent may be used in combination (combination therapy). Chemotherapy may be used alone to treat some types of cancer. Alternatively, chemotherapy may be used in combination with other types of treatment, for example, radiotherapy or alternative therapies (for example immunotherapy) as described herein. Additionally, a chemosensitizer may be administered as a combination therapy with a chemotherapy agent.

As used herein, a “chemotherapeutic agent” refers to a medicament that may be used to treat cancer, and generally has the ability to kill cancerous cells directly. Examples of chemotherapeutic agents include alkylating agents, antimetabolites, natural products, hormones and antagonists, and miscellaneous agents. Examples of alternate names are indicated in brackets. Examples of alkylating agents include nitrogen mustards such as mechlorethamine, cyclophosphamide, ifosfamide, melphalan (L-sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, 5FU), floxuridine (fluorodeoxyuridine, FUdR), cytarabine (cytosine arabinoside) and gemcitabine; purine analogs such as mercaptopurine (6-mercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, bleomycin, mitomycin (mitomycin C), idarubicin, epirubicin; enzymes such as L-asparaginase; and biological response modifiers such as interferon alpha and interlelukin 2. Examples of hormones and antagonists include luteinising releasing hormone agonists such as buserelin; adrenocorticosteroids such as prednisone and related preparations; progestins such as hydroxyprogesterone caproate, medroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Examples of miscellaneous agents include thalidomide; platinum coordination complexes such as cisplatin (cis-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as imatinib. Alternate names and trade-names of these and additional examples of chemotherapeutic agents, and their methods of use including dosing and administration regimens, will be known to a person versed in the art. In particular, suitable chemotherapeutic agents for use in accordance the invention include, without limitation, nanoparticle albumin-bound paclitaxels.

As used herein, the term “radiotherapeutic regimen” or “radiotherapy” refers to the administration of radiation to kill cancerous cells. Radiation interacts with various molecules within the cell, but the primary target, which results in cell death is the deoxyribonucleic acid (DNA). However, radiotherapy often also results in damage to the cellular and nuclear membranes and other organelles. DNA damage usually involves single and double strand breaks in the sugar-phosphate backbone. Furthermore, there can be cross-linking of DNA and proteins, which can disrupt cell function. Depending on the radiation type, the mechanism of DNA damage may vary as does the relative biologic effectiveness. For example, heavy particles (i.e. protons, neutrons) damage DNA directly and have a greater relative biologic effectiveness. Electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray.

Radiotherapy may further be used in combination chemotherapy, with the chemotherapeutic agent acting as a radiosensitizer. The specific choice of radiotherapy suited to an individual patient may be determined by a skilled person at the point of care, taking into consideration the tissue and stage of the cancer.

As used herein, the term “administer” or “administering” refers to introduce by any means a composition (e.g., a therapeutic agent) into the body of a mammal in order to prevent or treat a disease or condition (e.g., cancer).

As used herein, the terms “treating”, “treatment”, “therapy” and “therapeutic treatment” as used herein refer to curative therapy, prophylactic therapy, or preventative therapy. An example of “preventative therapy” is the prevention or lessening of a targeted disease (e.g., cancer) or related condition thereto. Those in need of treatment include those already with the disease or condition as well as those prone to have the disease or condition to be prevented. The terms “treating”, “treatment”, “therapy” and “therapeutic treatment” as used herein also describe the management and care of a mammal for the purpose of combating a disease, or related condition, and includes the administration of a composition to alleviate the symptoms, side effects, or other complications of the disease, condition. Therapeutic treatment for cancer includes, but is not limited to, surgery, chemotherapy, radiation therapy, gene therapy, and immunotherapy.

By “therapeutically effective amount” is meant an amount that relieves (to some extent, as judged by a skilled medical practitioner) one or more symptoms of the disease or condition in a mammal. Additionally, by “therapeutically effective amount” is meant an amount that returns to normal, either partially or completely, physiological or biochemical parameters associated with or causative of a disease or condition. A clinician skilled in the art can determine the therapeutically effective amount of a composition in order to treat or prevent a particular disease condition, or disorder when it is administered, such as intravenously, subcutaneously, intraperitoneally, orally, or through inhalation. The precise amount of the composition required to be therapeutically effective will depend upon numerous factors, e.g., such as the specific activity of the active agent, the delivery device employed, physical characteristics of the agent, purpose for the administration, in addition to many patient specific considerations. The determination of amount of a composition that must be administered to be therapeutically effective is routine in the art and within the skill of an ordinarily skilled clinician.

As used herein, the term “agent” or “drug” or “therapeutic agent” refers to a chemical compound, a mixture of chemical compounds, a biological macromolecule, or an extract made from biological materials such as bacteria, plants, fungi, or animal (particularly mammalian) cells or tissues that are suspected of having therapeutic properties. The agent or drug may be purified, substantially purified or partially purified. An “agent”, according to the present invention, also includes a radiation therapy agent.

As used herein, “modulation” or “increased” means that a desired/selected response is more efficient (e.g., at least 10%, 20%, 40%, 60% or more), more rapid (e.g., at least 10%, 20%, 40%, 60% or more), greater in magnitude (e.g., at least 10%, 20%, 40%, 60% or greater), and/or more easily induced (e.g., at least 10%, 20%, 40%, 60% or more) in the presence of an agent than in the absence of the agent.

As used herein, a “carrier” refers to any substance suitable as a vehicle for delivering an Active Pharmaceutical Ingredient (API) to a suitable in vitro or in vivo site of action. As such, carriers can act as an excipient for formulation of a therapeutic or experimental reagent containing an API. Preferred carriers are capable of maintaining an API in a form that is capable of interacting with a T cell. Examples of such carriers include, but are not limited to water, phosphate buffered saline, saline, Ringer's solution, dextrose solution, serum-containing solutions, Hank's solution and other aqueous physiologically balanced solutions or cell culture medium. Aqueous carriers can also contain suitable auxiliary substances required to approximate the physiological conditions of the recipient, for example, enhancement of chemical stability and isotonicity. Suitable auxiliary substances include, for example, sodium acetate, sodium chloride, sodium lactate, potassium chloride, calcium chloride, sorbitan monolaurate, triethanolamine oleate, and other substances used to produce phosphate buffer, Tris buffer, and bicarbonate buffer.

As used herein, the term “alternative therapeutic regimen” or “alternative therapy” may include for example, biologic response modifiers (including polypeptide-, carbohydrate-, and lipid-biologic response modifiers), toxins, lectins, antiangiogenic agents, receptor tyrosine kinase inhibitors (for example Iressa™ (gefitinib), Tarceva™ (erlotinib), Erbitux™ (cetuximab), imatinib mesilate (Gleevec™), proteosome inhibitors (for example bortezomib, Velcade™); VEGFR2 inhibitors such as PTK787 (ZK222584), aurora kinase inhibitors (for example ZM447439); mammalian target of rapamycin (mTOR) inhibitors, cyclooxygenase-2 (COX-2) inhibitors, rapamycin inhibitors (for example sirolimus, Rapamune™); farnesyltransferase inhibitors (for example tipifarnib, Zarnestra); matrix metalloproteinase inhibitors (for example BAY 12-9566; sulfated polysaccharide tecogalan); angiogenesis inhibitors (for example Avastin™ (bevacizumab); analogues of fumagillin such as TNP-4; carboxyaminotriazole; BB-94 and BB-2516; thalidomide; interleukin-12; linomide; peptide fragments; and antibodies to vascular growth factors and vascular growth factor receptors); platelet derived growth factor receptor inhibitors, protein kinase C inhibitors, mitogen-activated kinase inhibitors, mitogen-activated protein kinase kinase inhibitors, Rous sarcoma virus transforming oncogene (SRC) inhibitors, histonedeacetylase inhibitors, small hypoxia-inducible factor inhibitors, hedgehog inhibitors, and TGF-β signalling inhibitors. In particular, antiangiogenesis therapies are preferred alternative therapeutic regimens.

Furthermore, an immunotherapeutic agent would also be considered an alternative therapeutic regimen. Examples include chemokines, chemotaxins, cytokines, interleukins, or tissue factor. Suitable immunotherapeutic agents also include serum or gamma globulin containing preformed antibodies; nonspecific immunostimulating adjuvants; active specific immunotherapy; and adoptive immunotherapy. In addition, alternative therapies may include other biological-based chemical entities such as polynucleotides, including antisense molecules, polypeptides, antibodies, gene therapy vectors and the like. Such alternative therapeutics may be administered alone or in combination, or in combination with other therapeutic regimens described herein. Alternate names and trade-names of these agents used in alternative therapeutic regimens and additional examples of agents used in alternative therapeutic regimens, and their methods of use including dosing and administration regimens, will be known to a physician versed in the art. Furthermore, methods of use of chemotherapeutic agents and other agents used in alternative therapeutic regimens in combination therapies, including dosing and administration regimens, will also be known to a person versed in the art.

As used herein, a “medicament” is a composition capable of producing an effect that may be administered to a patient or test subject. The effect may be chemical, biological or physical, and the patient or test subject may be human, or a non-human animal, such as a rodent or transgenic mouse. The composition may include small organic or inorganic molecules with distinct molecular composition made synthetically, found in nature, or of partial synthetic origin. Included in this group are nucleotides, nucleic acids, amino acids, peptides, polypeptides, proteins, or complexes comprising at least one of these entities, The medicament may be comprised of the effective composition alone or in combination with a pharmaceutically acceptable excipient.

As used herein, a “pharmaceutically acceptable excipient” includes any and all solvents, dispersion media, coatings, antibacterial, antimicrobial or antifungal agents, isotonic and absorption delaying agents, and the like that are physiologically compatible. The excipient may be suitable for intravenous, intraperitoneal, intramuscular, intrathecal or oral administration. The excipient may include sterile aqueous solutions or dispersions for extemporaneous preparation of sterile injectable solutions or dispersion. Use of such media for preparation of medicaments is known in the art.

As used herein, a “pharmacologically effective amount” of a medicament refers to using an amount of a medicament present in such a concentration to result in a therapeutic level of drug delivered over the term that the drug is used. This may be dependent on mode of delivery, time period of the dosage, age, weight, general health, sex and diet of the subject receiving the medicament. The determination of what dose is a “pharmacologically effective amount” requires routine optimization which is within the capabilities of one of ordinary skill in the art.

As used herein, a “chemosensitizer” or “sensitizer” is a medicament that may enhance the therapeutic effect of a chemotherapeutic agent, radiotherapy treatment or alternative therapeutic regimen, and therefore improve efficacy of such treatment or agent. The sensitivity or resistance of a tumor or cancerous cell to treatment may also be measured in an animal, such as a human or rodent, by, e.g., measuring the tumor size, tumor burden or incidence of metastases over a period of time. For example, about 2, about 3, about 4 or about 6 months for a human and about 2-4, about 3-5, or about 4-6 weeks for a mouse. A composition or a method of treatment may sensitize a tumor or cancerous cell's response to a therapeutic treatment if the increase in treatment sensitivity or the reduction in resistance is about 10% or more, for example, about 30%, about 40%, about 50%, about 60%, about 70%, about 80%, or more, to about 2-fold, about 3-fold, about 4-fold, about 5-fold, about 10-fold, about 15-fold, about 20-fold or more, compared to treatment sensitivity or resistance in the absence of such composition or method. The determination of sensitivity or resistance to a therapeutic treatment is routine in the art and within the skill of a person versed in the art.

“Corresponding normal tissue” refers to the non-neoplastic tissue from which the cancer developed (“tissue of origin of the tumor”), either from the same or another mammal.

“Directly proportional” refers to a relation by a direct linear variation between two metrics.

Methods in accordance with the invention, wherein the level of methylation of the SPARC promoter is determined by any suitable method, including, e.g., methylation sensitive PCR, methylation-sensitive restriction digestion, bisulfite modification amplified by fluorescence-based real-time quantitative PCR, hybridization to oligonucleotide microarrays, and binding to anti-methylcytosine antibodies.

Bisulfite treatment of DNA converts unmethylated cytosine to uracil, while methylated cytosine does not react (Furuichi et al., Biochem. Biophys. Res. Commun. (1970) 41:1185-1191). Bisulfate modification of genomic DNA requires prior DNA denaturation because only methylcytosines that are located in single strands are susceptible to attack (Shapiro et al., J. Am. Chem. Soc. (1974) 96:206-212).

In bisulfite modification methylation detection processes, the most straightforward way of measuring methylation at CpG islands is by sequencing. In general, after denaturation and bisulfite modification of a genomic DNA sample, the resulting duplex DNA is obtained by primer extension and the fragment of interest is amplified by PCR techniques (Clark et al., Nucl. Acids Res. (1994) 22:2990-2997). Standard DNA sequencing of the PCR products then detects methylcytosine. Alternatively, one could clone the PCR products into plasmid vectors followed by sequencing of individual clones for a slowed method but one that could also provide methylation maps of single DNA molecules. In another variation, direct localization of methylcytosines in the product of bisulfite treatment instead of the PCR product can be done using only three deoxynucleotides (dATP, dCTP and dTTP) but lacking dGTP that produces an elongation stop at methylcytosine points (Radlinska and Skowronek, Acta Microbiol. Pol. (1998) 47:327-334).

Another process in the bisulfite class is methylation-specific PCR (Esteller et al., Cancer Res. (2001) 61:3225-3229 and Herman et al., Proc. Natl. Acad. Sci. USA (1996) 93:9821-9826), also called MSP. For example, in normal (non-cancerous) cells, cytosines in CpG islands are usually unmethylated, but they become methylated in the promoter sequences of genes associated with certain abnormal cellular processes, such as cancer (Esteller et al., Cancer Res. (1999) 59:793-797; Esteller et al., Cancer Res. (2001) 61:3225-3229; and Esteller et al., Hum. Mol. Genet. (2001) 10:3001-3007). Bisulfite-converted DNA strands are no longer complementary, so primer design in MSP is customized for each chain and methylation patterns of all sequences determined in separate reactions. MSP uses a difficult PCR process and critical primer designs using a narrow range of strand annealing temperatures, the PCR product is between 80 and 175 base pairs, each primer should contain at least two CpG pairs, the sense pair should contain a CpG pair at the 3′ end and primers contain non-CpG cytosines.

The MSP method was improved by combining methylation-specific PCR with in situ hybridization (Nuovo et al., Proc. Natl. Acad. Sci. USA 96:12754-12759, 1999) to allow for the methylation status of specific DNA sequences to be visualized in individual cells, for monitoring complex tissue samples having both tumor and normal cells.

Another method combines MSP with denaturing HPLC to allow for small cell mosaics of structurally normal or abnormal chromosomes to be detected (Baumer et al., Hum. Mutat. (2001)17:423-430). Specifically, following PCR amplification, the two alleles can be resolved from the two populations of PCR products by denaturing HPLC because they differ at several positions within the amplified sequence.

A quantification approach has been called “MethyLight” and uses fluorescent-based, real-time PCR (U.S. Pat. No. 6,331,393). The DNA is modified by the bisulfite treatment and amplified by fluorescence-based, real-time quantitative PCR using locus-specific PCR primers that flank an oligonucleotide probe with a 5′ fluorescence reporter dye and a 3′ quencher dye. The reporter is enzymatically released during the reaction, and fluorescence, which is proportional to the amount of PCR product and thus to the degree of methylation, can be sequentially detected in an automated nucleotide sequencer device.

A distinct approach has been to combine methyl-sensitive endonucleases with PCR amplification with subsequent hybridization to oligonucleotide microarrays (Huang et al., Hum. Mol. Genetics (1999) 8:459-70). In this case, methylation state was determined by digestion of unmethylated DNA using methylation sensitive restriction enzyme. Unmethylated DNA was enzymatically digested into fragments and did not generate amplicons after PCR whereas methylated DNA was protected from digestion and did generate amplicons after PCR. The presence or absence of amplicons was detected on oligonucleotide microarrays using fluorescent tags. Samples from normal tissues were used as a control with the supposition that these non-cancerous samples contained predominantly unmethylated cytosine residues. This procedure requires DNA from non-cancerous tissue to be available for use as an external control.

Another approach has been to perform a dual-hybridization assay using a test sample and an external reference sample known to be unmethylated in the analyzed region (Balog et al., Anal Biochem.(2002) 309: 301-310). In this case, a 190-bp DNA duplex was synthesized and used as an external reference sample, or DNA was obtained from a sample known to be unmethylated. The two samples were labeled with different fluorescent dyes, mixed and hybridized to an array containing 21 mer oligonucleotides. The external reference sample generated signal in a reference fluorescent channel on capture probes hybridizing to a thymidine residue. The presence of signal on a capture molecule probing for the presence of C within the test sample indicated methylation of that C residue. The process disclosed in U.S. Patent Application Publication No. 2006-0110741 for detection of DNA methylation at CpG sites using nucleic acid arrays and microarrays is a suitable methylation detection method for use in accordance with the invention. Specifically, this process allows for directly generating a reference sample from the sample to be tested and detecting methylation at large numbers of CpG island sites simultaneously. Further, the microarray process comprises dividing a DNA sample into two samples (a first sample and a second sample), amplifying the first DNA sample by a nucleic acid amplification process such that any methylcytosine residues are amplified as unmethylated cytosine residues, treating the amplified first sample and the (unamplified) second sample with bisulfite to convert unmethylated cytosine residues in both samples to deoxyuracil residues, labeling the bisulfite-converted second sample with a second fluorescent marker and the bisulfite-converted first sample with a first fluorescent marker, wherein the first and second fluorescent markers have non-overlapping fluorescent excitation and emission spectra; and hybridizing the first sample and the second sample onto a microarray device having a plurality of oligonucleotide capture probes designed to hybridize to CpG island sites of the DNA sample as converted and non-converted by bisulfite.

The invention provides methods of predicting resistance to chemotherapy in a mammal. In one embodiment, the invention provides methods of predicting resistance to chemotherapy in a mammal, wherein the resistance to chemotherapy in a mammal is predicted if CpG Dinucleotide Nos. of 1, 8, 9, and 16 are methylated in tumor tissue or if at least 3, preferably at least 5, more preferably at least 6 of CpG Dinucleotide Nos. of 1-2, 8-10, and 16-18 are methylated in tumor tissue.

The invention also provides methods of treating a mammal with a tumor. Accordingly, one preferred embodiment of the invention calls for the administration of a pharmaceutically-acceptable composition comprising a SPARC polypeptide is administered if CpG Dinucleotide Nos. of 1, 8, 9, and 16 are methylated in tumor tissue. Such a pharmaceutically-acceptable composition comprising a SPARC polypeptide can be administered in accordance with the invention if at least 3, preferably at least 5, more preferably at least 6 of CpG Dinucleotide Nos. of 1-2, 8-10, and 16-18 are methylated in tumor tissue. The invention further provides for the use of a demthylating agent to formulate a medicament, or to treat, a patient diagnosed as having a colorectal cancer characterized by methylation of a SPARC promoter.

Pharmaceutically-acceptable compositions that can be used in accordance with the invention include treating the mammal with a demethylating agent. Suitable demethylating agents include, e.g., 5-Aza-2′deoxycytidine, Procaine, Zebularine [1-(beta-D-ribofuranosyl)-1,2-dihydropyrimidin-2-one], and combinations thereof. Suitable mammals for treatment in accordance with the invention include, e.g., mammals suffering from or has been diagnosed with a colorectal cancer. In particular, wherein the mammal is a human.

Further, such demethylating agents can be used with suitable alternative therapeutic regimens include, without limitation, antibodies to molecules on the surface of cancer cells such as antibodies to Her2 (e.g., Trastuzumab), EGF or EGF Receptors, VEGF (e.g., Bevacizumab) or VEGF Receptors, CD20, and the like. The therapeutic agent may further comprise any antibody or antibody fragment which mediates one or more of complement activation, cell mediated cytotoxicity, inducing apoptosis, inducing cell death, and opsinization. For example, such an antibody fragment may be a complete or partial Fc domain.

By “antibodies” it is meant without limitation, monoclonal antibodies, polyclonal antibodies, dimers, multimers, multispecific antibodies (e.g., bispecific antibodies). Antibodies may be murine, human, humanized, chimeric, or derived from other species. An antibody is a protein generated by the immune system that is capable of recognizing and binding to a specific antigen. A target antigen generally has numerous binding sites, also called epitopes, recognized by CDRs on multiple antibodies. Each antibody that specifically binds to a different epitope has a different structure. Thus, one antigen may have more than one corresponding antibody.

An antibody includes a full-length immunoglobulin molecule or an immunologically active portion of a full-length immunoglobulin molecule, i.e., a molecule that contains an antigen binding site that immunospecifically binds an antigen of a target of interest or part thereof. Targets include cancer cells or other cells that produce autoimmune antibodies associated with an autoimmune disease.

The immunoglobulins disclosed herein can be of any class (e.g., IgG, IgE, IgM, IgD, and IgA) or subclass (e.g., IgG1, IgG2, IgG3, IgG4, IgA1 and IgA2) of immunoglobulin molecule. The immunoglobulins can be derived from any species.

“Antibody fragments” comprise a portion of a full length antibody, which maintain the desired biological activity. “Antibody fragments” are generally the antigen binding or variable region thereof. Examples of antibody fragments include Fab, Fab′, F(ab′)2, and Fv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The monoclonal antibodies referenced herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567). Chimeric antibodies of interest herein include “primatized” antibodies comprising variable domain antigen-binding sequences derived from a non-human primate (e.g., Old World Monkey or Ape) and human constant region sequences.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express Fc.γ.RIII only, whereas monocytes express FcγRI, FcγRII and FcγRIII. To assess ADCC activity of a molecule of interest, an in vitro ADCC assay may be performed (U.S. Pat. No. 5,003,621; U.S. Pat. No. 5,821,337). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al PNAS (USA), 95:652-656 (1998).

An antibody which “induces cell death” is one which causes a viable cell to become nonviable. Cell death in vitro may be determined in the absence of complement and immune effector cells to distinguish cell death induced by antibody-dependent cell-mediated cytotoxicity (ADCC) or complement dependent cytotoxicity (CDC). Thus, the assay for cell death may be performed using heat inactivated serum (i.e., in the absence of complement) and in the absence of immune effector cells. To determine whether the antibody is able to induce cell death, loss of membrane integrity as evaluated by uptake of propidium iodide (PI), trypan blue or 7AAD can be assessed relative to untreated cells. Cell death-inducing antibodies are those which induce PI uptake in the PI uptake assay in BT474 cells.

An antibody which “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies).

Administer means to deliver a therapeutic formulation to the patient through any suitable route including, without limitation, intravenous, intraperitoneal, intratumoral, or inhalational. Formulations suitable for administration via inhalation include aerosol formulations. The aerosol formulations can be placed into pressurized acceptable propellants, such as dichlorodifluoromethane, propane, nitrogen, and the like. They also can be formulated as non-pressurized preparations, for delivery from a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind previously described. In this regard, the formulation desirably is suitable for intratumoral administration, but also can be formulated for intravenous injection, intraperitoneal injection, subcutaneous injection, and the like.

Formulations suitable for anal administration can be prepared as suppositories by mixing the active ingredient with a variety of bases such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

In addition, the composition of the invention can comprise additional therapeutic or biologically-active agents. For example, therapeutic factors useful in the treatment of a particular indication can be present. Factors that control inflammation, such as ibuprofen or steroids, can be part of the composition to reduce swelling and inflammation associated with in vivo administration of the pharmaceutical composition and physiological distress.

The following examples further illustrate the invention but, of course, should not be construed as in any way limiting its scope.

### Examples

Cell Lines and Clinical Samples:

Human colorectal cancer (CRC) cell lines, MIP 101, RKO, HCT116 and HT29 (ATCC), and a normal colon cell line CCD 112CON (ATCC), were maintained in DMEM supplemented with 10% fetal bovine serum; 1% Kanamycin, Streptomycin, Penicillin; and incubated at 37° C. and 5% CO₂. To determine the effect of reversing the methylation status of SPARC, a demethylating agent (5-Aza-2′deoxycytidine, “5-Aza”) was added to the culture media at a final concentration of 4 μm for a period of 7 days with medium changes on day 4, before assessing its effect on cell viability (WST-1), apoptosis (Caspase 3/7 and TUNEL) and cell proliferation.

DNA Isolation:

Genomic DNA of clinical samples were extracted by phenol chloroform after isolation from paraffin sections by laser capture microdissection (Molecular Machine & Industries, jxCut Laser Microdissection). Briefly, microdissected materials were digested overnight at 42° C. (digestion buffer: 10 mM Tris-HCl pH 8, 1 mM EDTA, 1% Tween20, 0.5% Proteinase K). Samples were heated to 95° C. for 10 min to inactivate the enzyme. One volume of phenol chloroform isoamyl alcohol (25:24:1) was mixed by inversion with the digested samples and added to the Phase Lock Gel™ Eppendorf tubes, previously spun to provide a uniform gel surface. After spinning at 14,000×g for 5 min at room temperature, the aqueous layer was removed, placed into a new tube, glycogen (20 mg/ml) was added, and the solution was mixed by inversion. Precipitation of the DNA was performed with 3 volumes of 95% ethanol, 0.1M NaOAc for 2 hours at −20° C. and then centrifuged at 14,000×g for 10 min at room temperature. DNA pellets were washed with 70% ethanol and spun at 14,000×g for 10 min, air dried and resuspended in 10 μl of modified TE (10 mM Tris [pH8]/0.1mM EDTA).

Methylation-Specific PCR (MSP) (Bisulfite Sequencing) and RT-PCR:

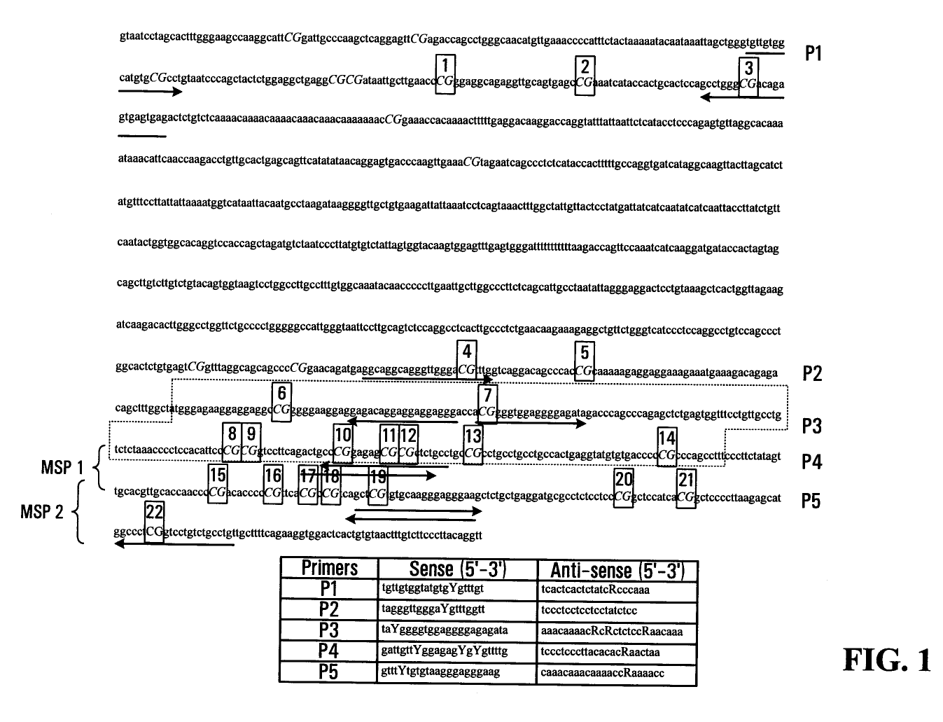

This technique is based on bisulfite treatment of the DNA, which modifies cytosines to uracil except when they are methylated (5-methylcytosine), followed by PCR. After bisulfite modification the DNA was amplified using methylation specific primers. Two primer sets, previously described, were used to target the CpG island located in the putative promoter region of the SPARC gene, and are referred as MSP1 and MSP2. Primers targeting INK4A(p16) were used as control. Direct DNA sequencing was also performed after bisulfite treatment with primers covering the CpG region of SPARC as shown in FIG. 1, 1,500 bp upstream and including exon 1. For RT-PCR, mRNA isolated from CRC cell lines were extracted using Trizol (Invitrogen). Primers to amplify SPARC, sense 5′-GGGAGGGCCTTGGCAG-3′ antisense 5′-GCACATGGGGGTGTTG-3′ and actin, sense 5′-GCCACGGCTGCTTCCAG-3′antisense 5′-GGCGTACAGGTCTTTGC-3′ were annealed at 49° C. in 45 cycles after reverse transcription for 1 hour at 50° C. Amplicons were separated in 2.5% agarose gel electrophoresis.

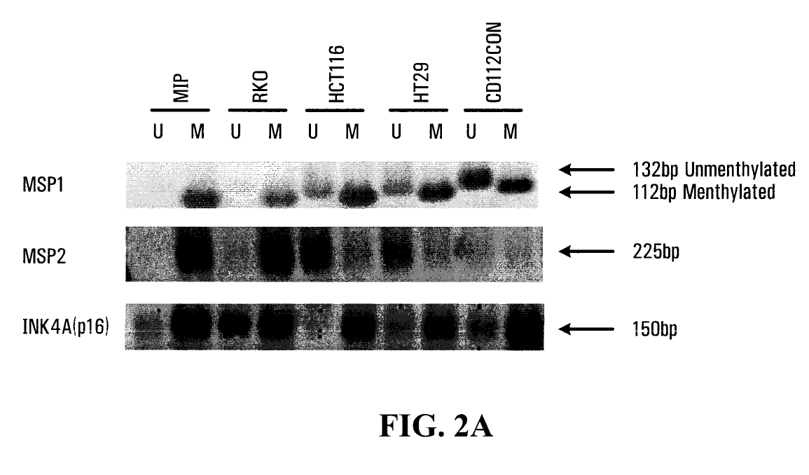

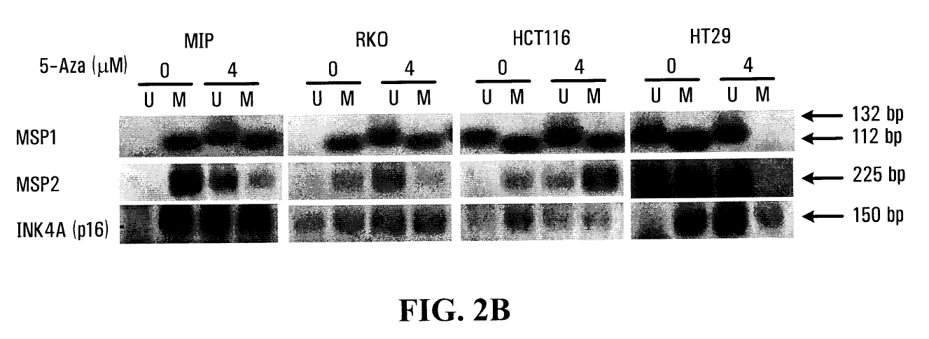

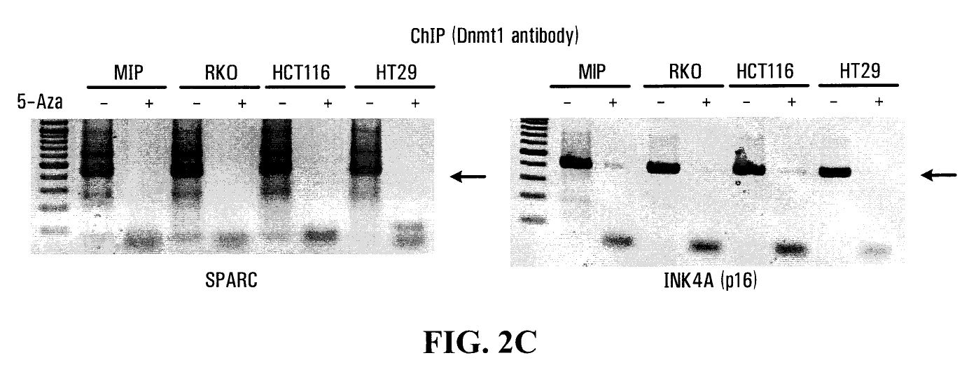

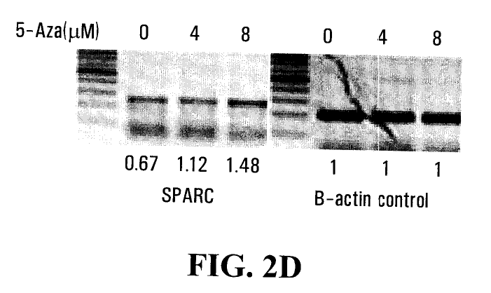

FIG. 1 shows a map of the CpG island in the promoter region at 5′ of the SPARC gene, including regions flanked by the bisulfite sequencing primers (P1-P5, arrows and sequences), numbered CG positions within the sequencing region (grey), exon 1 (boxed). Brackets show regions amplified using methylation specific primers MSP1 and MSP2. FIG. 2 depicts the use of methylation Specific PCR (MSP). Primers targeting the promoter-exon 1 region of SPARC were used to determine its methylation status in: a) CRC cell lines (MIP101, RKO, HCT116, HT29) and normal colon (CCD-112CoN); and b) CRC cell lines after treatment with 5-Aza for 7 days.

Cell Viability, Proliferation and Clonogenic Assay:

Cell viability was evaluated using WST-1 (Roche) following the manufacturer's instructions. Cells exposed to 5-Aza for a period of 7 days (and untreated controls) were seeded at 10,000 cells per well in triplicate into a 96-well plate. Twelve hours later, 5-Fluorouracil (5-FU) was added to a final concentration of 0, 500 or 1,000 μM for an additional 24 h. WST reagent was added to each well and incubated at 37° C. after which plates were read at 60 min at 440 nm. Three independent experiments were analyzed. The significance of differences were determined using Student's t-test (P<0.05).

To determine the rate of cell proliferation, cells exposed to 5-Aza for 7 days (and untreated controls) were seeded at 20,000 cells/well into a 48-well plate. Cells were counted from duplicate wells at 24, 48 and 72 h. The effect of 5-FU exposure for 24 hour-period on cell proliferation in the presence or absence of 5-Aza was also determined. We performed two independent experiments.

For the clonogenic assay, 5-Aza treated and untreated control cells were seeded at 1,000 cells/per well in a 48-well plate. Twenty-four hours later, cells were incubated with 500-1,000 μM 5-FU for 7 days, with a change in media and drug on day 4. Cells were then stained with 0.2% crystal violet. The number of colonies in the treated group was calculated based on the colonies formed from the control, untreated cells. Assays were performed in triplicate.

Apoptosis Assay:

Apoptosis was evaluated by Caspase 3/7 assay (Promega). Total cell lysate protein (20 μg) were diluted with 1:1 substrate. Relative luminescence units (RLU) were quantified using a Viktor 1420 Multilabel counter (Perkin Elmer). Significant differences were determined using Student's t-test (P<0.05). Apoptosis was also assessed using the terminal deoxiribonucleotidyl treansferase-mediated dUTP nick end labeling assay (TUNEL) that is well known in the art. Both 5-Aza treated and untreated cells were seeded into a 48-well plate at 100,000 cells per well. Twelve hours later 5-FU was added to these cells at a final concentration of 1,000 μM for an additional 24 hours. Supernatant and trypsinized cells were collected and attached to microscope slides using cytospin (Shandon) at 3,000 rpm for 10 min. After fixation in 4% paraformaldehyde at 4° C. for 20 min and 2 washes in PBS, slides were processed for TUNEL assay (Promega) as indicated by the manufacturer. Apoptotic cells were counted in 3 random fields at 40× magnification in triplicate independent experiments. Total number of cells per field (greater than 100) was determined by based on DAPI nuclear staining.

Chromatin Immunoprecipitation (ChIP) Assay:

To evaluate the effect of 5-Aza exposure on the interaction between Dnmtl (a DNA methyltranferase) with the promoter region of SPARC, a ChIP assay was performed using a ChIP kit (Epigentek) as per the manufacturer's instructions. Cells were fixed with formaldehyde for protein/DNA crosslinking and lysed. The DNA was sheared by sonication (10 pulses, 30 minutes on 30 minutes off) and added to a well coated with the antibody to the protein of interest (anti-Dnmtl, cat. #A-1001, Epigentek). Washes were performed to remove unbound material, while Dnmtl-bound DNA was released by protein digestion with proteinase K. The DNA was purified through a column and PCR was performed using primers designed to target the SPARC promoter region.

### Example 1

This Example demonstrates the hypermethylation of the SPARC promoter region in colorectal cancer cell lines.

The methylation status of the SPARC promoter (see FIG. 1) was determined by methylation specific PCR in four CRC cell lines (MIP101, RKO, HT29, and HCT 116) and one normal colon cell line (CCD 112CoN) (FIG. 2a). Hypermethylation was observed in the entire promoter region of MIP 101 and RKO cells flanked by MSP1 and MSP2. In HCT116 and HT29 cells, partial methylation was noted with methylated CpG sites in the MSP1 region, but not in the MSP2 region. The normal colon cell line CCD-112CoN also showed partial methylation, with a methylated MSP1 region and an unmethylated MSP2 region (FIG. 2a). In addition, as a control, the methylation status of the promoter of the INK4A(pl6) gene was also assessed. INK4A(pl6) gene has been well described as hypermethylated in some colon cancers, and was found to be hypermethylation in MIP101 and HCT 116 cells, but only partial methylation in RKO and HT 29 CRC cells.

### Example 2

This Example demonstrates that the effects of exposure to a demethylating agent on the methylation status of the SPARC promoter region in colorectal cancer cell lines.

Exposure to a demethylating agent, 5-Aza (4 μM), for 7 days in vitro resulted in a change in the methylation status in the MSP1 and MSP2 regions of the promoters for both SPARC and INK4A(pl6) in all four CRC cell lines (FIG. 2b). Unmethylated regions were detected after incubation with 5-Aza, especially in those cell lines where there was complete methylation in both MSP regions, such as in MIP101 and RKO cells. Reversal of hypermethylation within the region of the SPARC promoter by 5-Aza was confirmed by ChIP assays (FIG. 2c). In all CRC cell lines, an interaction between the SPARC promoter with Dnmtl (a DNA methyltransferase that catalyzes the transfer of a methyl group to DNA, resulting in DNA methylation) could be detected (FIG. 2c). However, following exposure to 5-Aza, this interaction was no longer observed.

### Example 3

This Example demonstrates that hypermethylation of the SPARC promoter was more commonly observed in human colon cancers than in the normal colon.

Methylation specific PCR was used initially to determine the methylation status of DNA isolated from laser-capture microdissected specimens of human colorectal cancers and normal colon. The clinical characteristics of the specimens studied is shown in the following table:

| AJCC | Age range | Gender |
|---|---|---|
| staging | (mean) | (M/F) |
| I | 44-81 (62.5) | 3/2 |
| II | 58-73 (65.5) | 2/0 |
| III | 55-75 (61.7) | 3/0 |
| IV | — | — |
| Normal | 34-68 (54.2) | 3/2 |

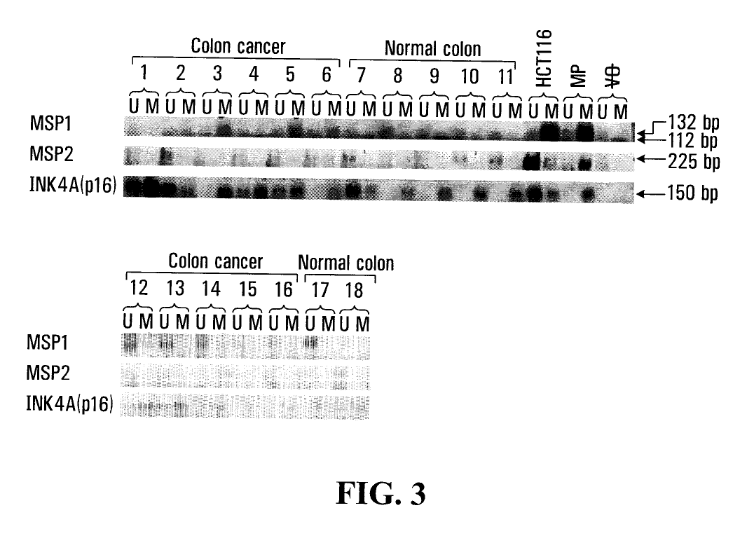

It was observed that the promoter for SPARC was methylated in 4 of 10 human colon cancers while no methylation was observed in any of the 5 normal colon samples in the regions assessed by MSP1 and MSP2. FIG. 3 shows methylation pattern of the SPARC promoter from primary colorectal cancers and normal colon were assessed by MSP. Methylation of the SPARC promoter was observed in 4 of 10 colon cancers while no methylation was observed in the normal colon.

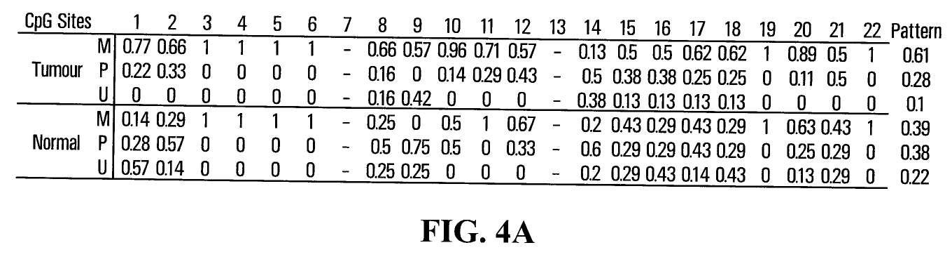

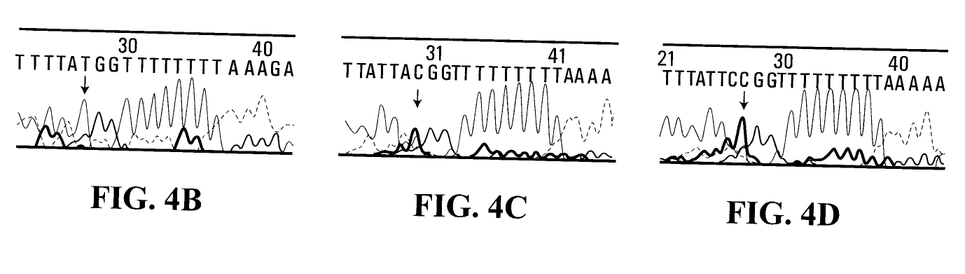

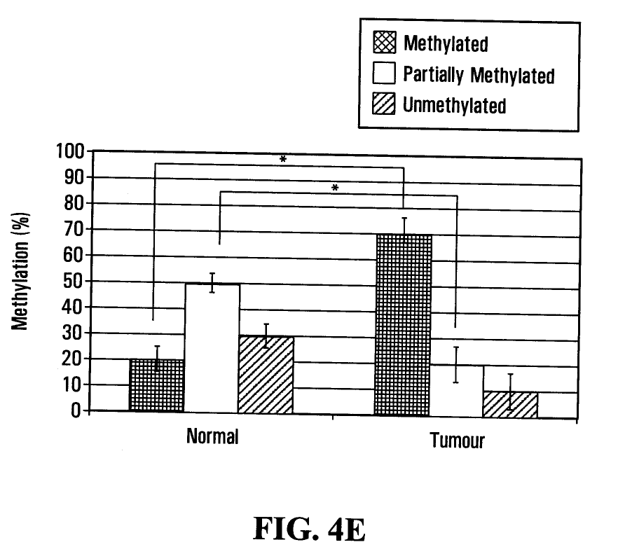

To further demonstrate the methylation status of the CpG regions within the 1,500 bp upstream of the SPARC promoter and including exon 1. DNA from the same microdissected clinical samples were subjected to bisulfate treatment followed by DNA sequencing. FIG. 4 shows the results of direct bisulfite DNA sequencing of human colorectal cancers and normal colon in the 1,500 bp region 5′ of the SPARC gene (including exon 1-intron 1) a) The frequency of M=methylation, P=partial methylation, and U=unmethylation at each CpG site (numbers correspond to grey boxes in FIG. 1) is provided for colorectal cancers (tumors) and normal colon.

There was variability in the methylation status at various potential sites. The CpG positions 1-2, 8-10, 16-18 were more frequently methylated in colorectal cancer. In normal colon, the regions that were consistently unmethylated were CpG positions 1, 8, 9 and 16, which were mostly methylated in colorectal cancer (FIG. 4a).

Overall, a significantly higher proportion of CpG sites were methylated within the SPARC promoter in colorectal cancers (60%), while only 35% of the sites were methylated in normal colon (p=0.03) (See FIG. 4a, e). The proportion of partially methylated sites were significantly lower in the colorectal cancers (28%) compared to the normal colon (46%) (p=0.04). In the normal colon, there appeared to be a similar frequency of CpG sites that were either completely methylated, partially or completely unmethylated (FIG. 4b, c, d). A higher percentage of methylated CpG sites within the SPARC promoter region was detected in clinical samples of colorectal cancers than in the normal colon (FIG. 4e).

When characteristics such as age, gender and stage of disease were evaluated in relation to the methylation state of the SPARC promoter within specific CpG sites (positions 1-2, 8-10, 16-18), the following were observed: (1) hypermethylation increased with patient age in both groups (colorectal and normal colon), but a greater proportion was observed in colorectal cancers; and (2) the most advanced colorectal cancers had the highest proportion of methylated sites within the SPARC promoter than either the earlier stage or normal colon. Examination of the number of colorectal cancers had greater than 50% methylated CpG sites revealed that 80% of colorectal cancer (8 of 10 samples) met this criteria, while only 20% of normal colons (1 of 5 samples) could be considered as hypermethylated.

### Example 4

This Example demonstrates that the exposure to demethylating agent, 5-Aza-2′deoxycytidine, increases the sensitivity of CRC cells with methylated SPARC promoter to chemotherapy

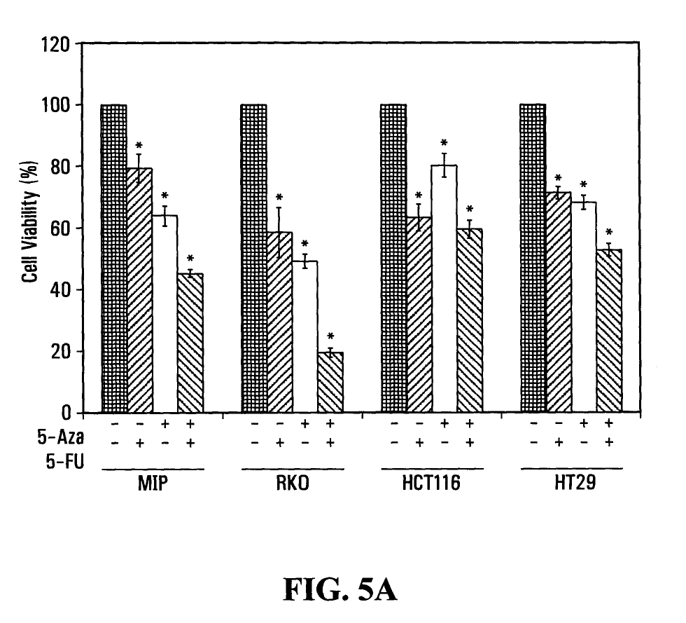

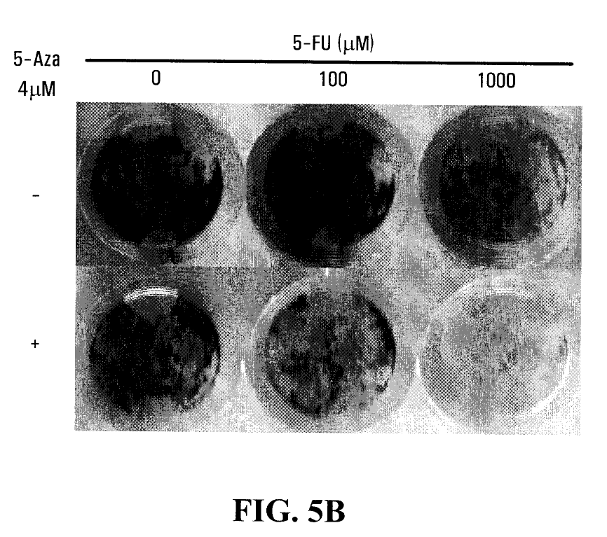

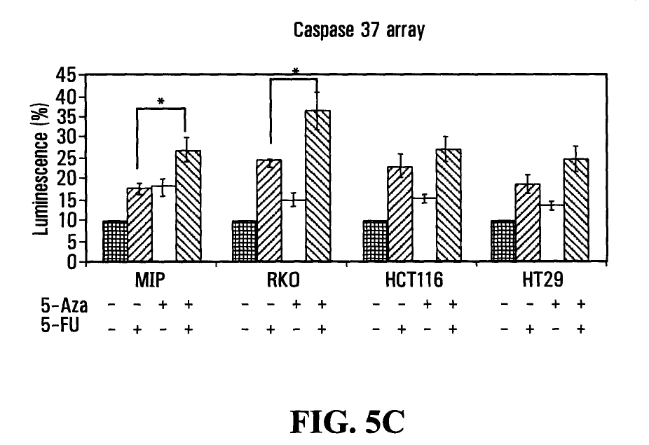

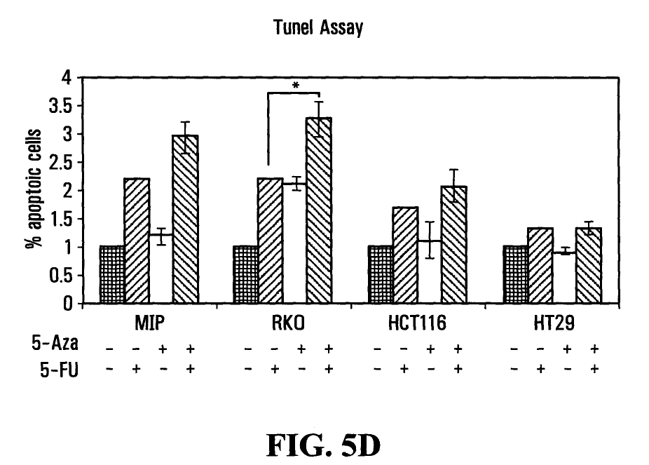

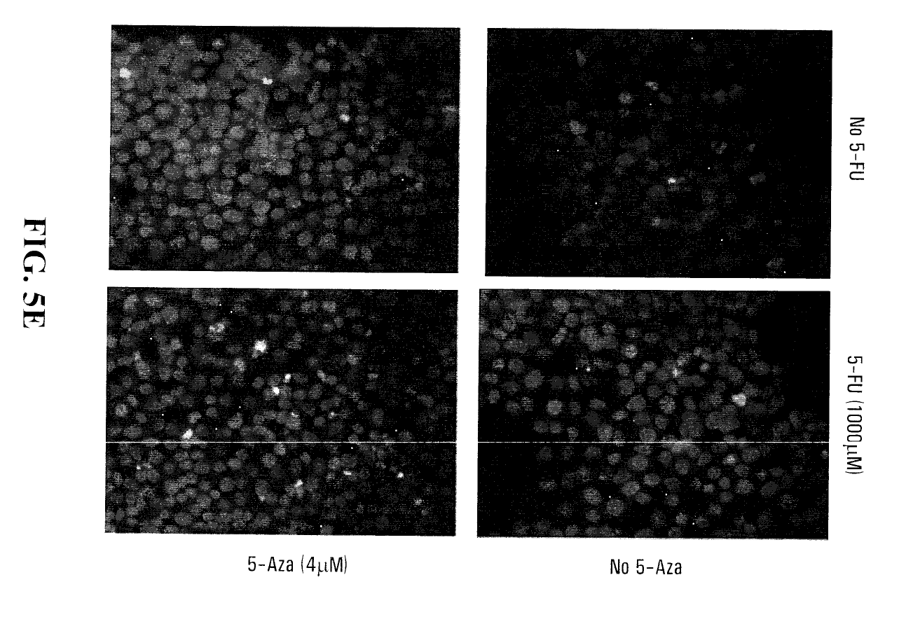

FIG. 5a depicts the effect of exposing CRC cells to 5-Aza, a demethylating agent, on their sensitivity to 5-FU. Cell viability was assessed following a 7-day preincubation with 4 μM 5-Aza and 24-hr exposure to incremental concentrations of 5-FU. Increased sensitivity to 500 and 1,000 μM 5-FU was observed following pre-incubation with 5-Aza in MIP 101 cells (p<0.05) (FIG. 5a).

The effect of 5-Aza in combination with 5-FU on apoptosis was also assessed by clonogenic assay of MIP 101 cells following pre-incubation with 5-Aza and incremental concentrations of 5-FU showed enhanced sensitivity in cells pre-incubated 5-Aza (FIG. 5b).

As noted above, hypermethylation of the SPARC promoter could be reversed following incubation with 5-Aza, which also resulted in higher SPARC expression (FIG. 2d). Therefore, we next examined if pre-incubation with 5-Aza would influence the sensitivity of MIP101, RKO, HCT116 and HT29 CRC cells to 5-FU.

In MIP 101 cells, pre-incubation with 5-Aza resulted in a significant decrease in cell viability when cells were subsequently treated with either 500 or 1,000 μM of 5-FU in comparison to 5-FU alone: from 87.74%+12.8 viable cells (500 μM 5-FU only) to 53.52%+12.7 (p=0.03) and 82.67%+7.4 viable cells (1,000 μM 5-FU only) to 47.56%+4.91 (p=0.002). In RKO cells and the other CRC cells with partial methylation of the SPARC promoter, such as HT116 and HT29, pre-incubation with 5-Aza did not significantly change the effect of 5-FU on cell viability (FIG. 5a). This enhanced sensitivity of MIP101 cells to 5-FU following pre-incubation with 4 μM 5-Aza was also supported by the results of clonogenic assays, which revealed fewer colonies in the groups exposed to both 5-Aza and 5-FU (FIG. 5b).

The decrease in cell viability of MIP101 cells following 5-FU treatment that resulted from pre-incubation with 5-Aza correlated with an increase in apoptosis, as significantly higher levels of caspase 3/7 were detected in MIP101 cells that were subjected to the same experimental conditions as those that resulted in decreased cell viability (pre-incubation with 4 μM 5-Aza followed by a 24-hr treatment with 1,000 μM 5-FU). Similarly, in RKO cells, which also showed complete hypermethylation of the SPARC promoter, there were higher levels of caspase 3/7 following pre-incubation with 5-Aza and subsequent treatment with 5-FU (FIG. 5c). These observations were also confirmed by noting significantly greater numbers of TUNEL-positive cells in MIP101 and RKO cells similarly exposed to 5-Aza and 5-FU (FIG. 5d, e).

### Example 5

This Example demonstrates that cell proliferation decreases in CRC cell lines following incubation with 5-Aza, but that this reduction in proliferation does not protect the cells from 5-FU.

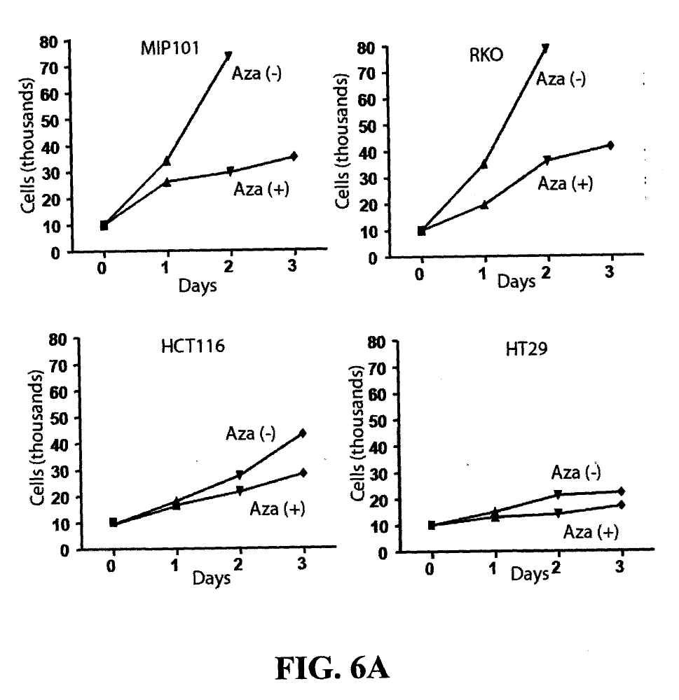

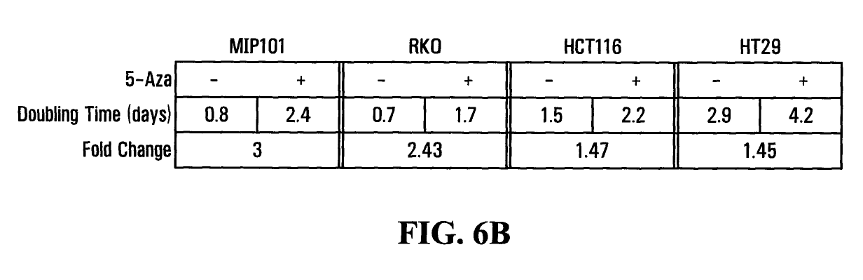

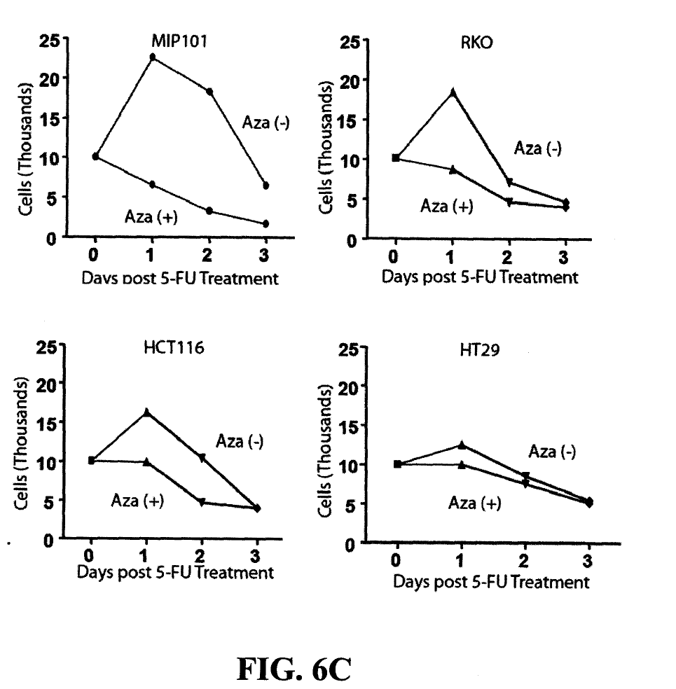

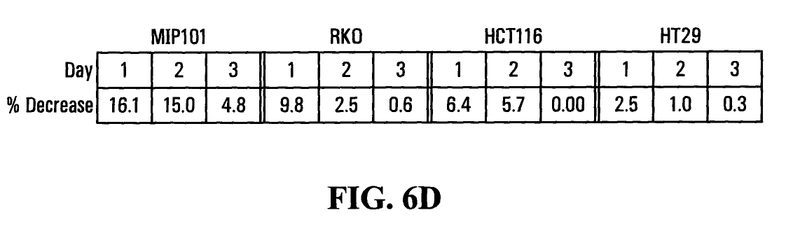

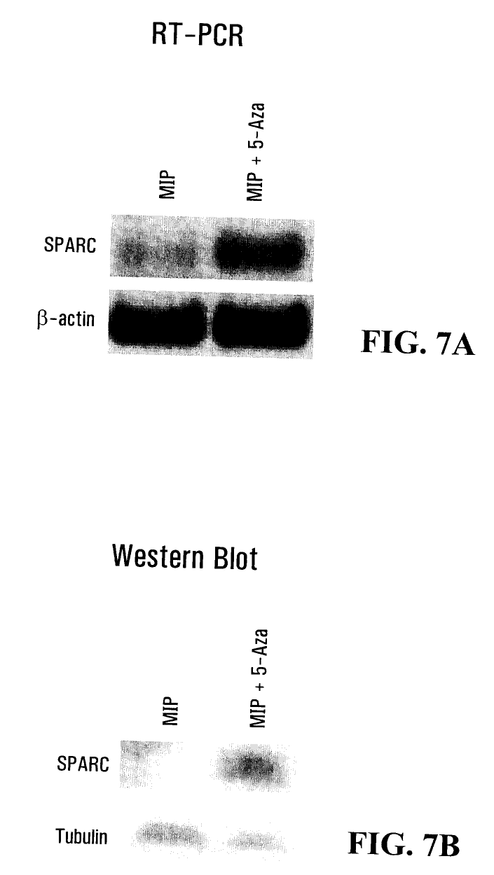

FIGS. 6a, b show the effect of exposure of CRC cell lines to 5-Aza on cell proliferation. Exposure to 5-Aza affected the cell proliferation in all 4 cell lines (MIP101, RKO, HCT 116, and HT29) (broken lines are the 5-Aza treated cells, solid lines control), with the greatest reduction in growth observed with MIP101 and RKO, resulting in a 3- and 2.4-fold increase in cell doubling time. In combination with 5-FU, cell proliferation decreased in all four CRC cell lines, with the greatest effect occurring within the first day of 5-FU treatment in MIP101 and RKO cells (FIG. 6c, d; broken lines are the 5-FU/5-Aza treated cells, solid lines control).

The most dramatic decrease in cell proliferation following incubation with 4 μM 5-Aza was again noted with MIP101 and RKO cells, which have complete methylation of the SPARC promoter, beginning as early as 24 hrs of incubation (FIG. 6a, b), with a 3- and 2.43-fold increase in doubling time (from 0.8 days to 2.4 days, and 0.7 to 1.7 days) in MIP101 and RKO respectively (FIG. 6b). In HCT 116 and HT29 cells with partial methylation of the SPARC promoter, a less dramatic yet significant decrease in cell proliferation was also observed when these cells were incubated with 5-Aza, which was most noticeable after 2-days (FIG. 6a, b).

Exposure to 5-FU, in addition to 5-Aza, resulted in a steady decline in cell numbers in all cell lines, but more dramatically within 24 hrs, in the MIP101 and RKO cells in comparison to HCT116 and HT 29 cells (FIG. 6c, d).

RT-PCR and Western blot analysis of MW101 cells confirms that 5-Aza causes reactivation of SPARC RNA and protein expression.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

SEQ ID NO: 1

gtaatcctagcactttgggaagccaaggcattCGgattgcccaagctca
ggagttCGagaccagcctgggcaacatgttgaaaccccatttctactaa
aaatacaataaattagctgggtgttgtggcatgtgCGcctgtaatccca
gctactctggaggctgaggCGCGataattgcttgaaccCGggaggcaga
ggttgcagtgagcCGaaatcataccactgcactccagcctgggCGacag
agtgagtgagactctgtctcaaaacaaaacaaaacaaacaaacaaaaaa
acCGgaaaccacaaaactttttgaggacaaggaccaggtatttattaat
tctcatacctcccagagtgttaggcacaaaataaacattcaaccaagac
ctgttgcactgagcagttcatatataacaggagtgacccaagttgaaaC
Gtagaatcagccctctcataccactttttgccaggtgatcataggcaag
ttacttagcatctatgtttccttattattaaaatggtcataattacaat
gcctaagataaggggttgctgtgaagattattaaatcctcagtaaactt
tggctattgttactcctatgattatcatcaatatcatcaattccttatc
tgttcaatactggtggcacaggtccaccagctagatgtctaatccctta
tgtgtctattagtggtacaagtggagtttgagtgggattttttttttta
agaccagttccaaatcatcaaggatgataccacagtagcagcttgtctt
gtctgtacagtggtaagtcctggccttgcctttgtggcaaatacaaccc
ccttgaattgcttggcccttctcagcattgcctaatattagggaggact
cctgtaaagctcactggtagaagatcaagacacttgggcctggttctgc
ccctgggggccattgggtaattccttgcagtctccaggcctcacttgcc
ctctgaacaagaaagaggctgttctgggtcatccctccaggcctgtcca
gcctggcactctgtgagtCGgtttaggcagcagcccCGgaacagatgagg
caggcagggttgggaCGtttggtcaggacagcccacCGcaaaaagagga
ggaaagaaatgaaagacagagacagctttggctatgggagaaggaggagg
cCGggggaaggaggagacaggaggaggagggaccaCGgggtggagggga
gatagacccagcccagagctctgagtggtttcctgttgcctgtctctaa
acccctccacattccCGCGgtccttcagactgccCGgagagCGCGctct
gcctgcCGcctgcctgcctgccactgaggtatgtgtgaccccCGcccag
cctttcccttctatagttgcacgttgcaccaacccCGacaccccCGttc
aCGcCGtcagctCGtgtgcaagggagggaagctctgctgaggatgcgcc
tctcctccCGgctccatcaCGgctccccttaagagcatggccctCGgtc
ctgtctgcctgttgcttttcagaaggtggactcactgtgtaactttgtc
ttcccttacaggtt

1. A method for assessing the resistance to chemotherapy of a tumor in a mammal comprising:
(a) determining the level of SPARC promoter methylation in the tumor tissue; and
(b) assessing the resistance to chemotherapy of the tumor,
wherein the resistance to chemotherapy of the tumor is directly proportional to the level of SPARC promoter methylation in the tumor tissue.

2. A method for assessing the resistance to chemotherapy of a tumor in a mammal comprising:
(a) determining the level of SPARC promoter methylation in the tumor tissue;
(b) determining the level of SPARC promoter methylation in the corresponding normal tissue; and
(c) assessing the resistance to chemotherapy of the tumor,
wherein the resistance to chemotherapy of the tumor is directly proportional to the difference between the level of methylation of the SPARC promoter in the tumor tissue and the level of methylation of the SPARC promoter in the corresponding normal tissue.

3. The method for assessing the resistance to chemotherapy of a tumor in a mammal according to claim 2
wherein determining the level of SPARC promoter methylation comprises determining methylation status of the cytosines at the following positions in SEQ ID NO:1:
186
210
240
1099
1120
1184
1218
1296
1298
1315
1322
1324
1336
1372
1414
1423
1429
1432
1440
1485
1496
1521

4. The method of claim 3, wherein the resistance to chemotherapy in a mammal is predicted if at least 3 of the cytosines at the following positions in SEQ ID NO: 1
186
210
1296
1298
1315
1423
1429
1432
are methylated in tumor tissue but not the corresponding normal tissue.

5. The method of claim 3, wherein the resistance to chemotherapy in a mammal is predicted if the cytosines at the following positions in SEQ ID NO: 1
186
1296
1298
1423
are methylated in tumor tissue.

6. The method of claim 3, wherein the resistance to chemotherapy in a mammal is predicted if at least 5 of the cytosines at the following positions in SEQ ID NO: 1
186
210
1296
1298
1315
1423
1429
1432
are methylated in tumor tissue.

7. The method of claim 3, wherein the corresponding normal tissue is from the same mammal as the mammal with the tumor.

8. The method of claim 3, wherein the corresponding normal tissue is from a different mammal from the mammal with the tumor.

9. The method of claim 1, wherein the level of methylation of the SPARC promoter is determined by a method comprising at least one of methylation sensitive PCR, methylation-sensitive restriction digestion, bisulfate modification amplified by fluorescence-based real-time quantitative PCR, hybridization to oligonucleotide microarrays, and binding to anti-methylcytosine antibodies.

10. A method of treating a mammal with a tumor, wherein said method comprises performing the method of claim 2
and
administering SPARC polypeptide and/or a demethylating agent to the mammal based on the level of SPARC promoter methylation in the tissue.

11. The method of claim 10, wherein the composition comprising a SPARC polypeptide is administered if at least 3 of the cytosines at the following positions in SEQ ID NO: 1
186
1296
1298
1423
are methylated in tumor tissue.

12. The method of claim 10, wherein the composition comprising a SPARC polypeptide is administered if at least 5 of the cytosines in the following positions of SEQ ID NO:1
186
210
1296
1298
1315
1423
1429
1432
are methylated in tumor tissue.

13. The method of claim 10, wherein the composition comprising a demethylating agent is administered if at least 3 of the cytosines at the following positions in SEQ ID NO: 1
186
1296
1298
1423
are methylated in tumor tissue.

14. The method of claim 10, wherein the composition comprising a demethylating agent is administered if at least 5 of the cytosines at the following positions in SEQ ID NO: 1
186
210
1296
1298
1315
1423
1429
1432
are methylated in tumor tissue.

15. The method of claim 10, wherein a demethylating agent is administered if at least 5 of CpG Dinucleotide Nos. of 1-2, 8-10, and 16-18 are methylated in tumor tissue.

16. (canceled)

17. The method of claim 10, wherein the demethylating agent is selected from the group consisting of 5-Aza-2′deoxycytidine, Procaine, Zebularine [1-(beta-D-ribofuranosyl)-1,2-dihydropyrimidin-2-one], and combinations thereof.

18. The method of claim 1, wherein the mammal is suffering from or has been diagnosed with a colorectal cancer.

19. The method for assessing the resistance to chemotherapy of a tumor in a mammal according to claim 1, comprising
determining ex vivo the level of SPARC promoter methylation in a tumor tissue sample obtained from the tumor.

20. The method for assessing the resistance to chemotherapy of a tumor in a mammal according to claim 2, comprising
determining ex vivo the level of SPARC promoter methylation in a tumor tissue sample obtained from the tumor.

21-36. (canceled)

37. The method according to claim 2, wherein the mammal is suffering from or has been diagnosed with a colorectal cancer.

38. The method according to claim 10, wherein the mammal is suffering from or has been diagnosed with a colorectal cancer.
